(19) United States
(12) Patent Application Publication
(10) Pub. No.: US 2009/0127226 A1
(43) Pub. Date: May 21, 2009

(54) PROCESS FOR PRODUCING SELF-SUPPORTING TITANIUM AND NICKEL LAYERS

(75) Inventors: Eckhard Quandt, Heikendorf (DE); Holger Rumpf, Reutlingen (DE); Christiane Zamponi, Kiel (DE)

Correspondence Address: MARSHALL, GERSTEIN & BORUN LLP (FISHER), 233 SOUTH WACKER DRIVE, 6300 SEARS TOWER, CHICAGO, IL, 60606, US

(73) Assignee: Arcadis GMGH & Co. KG, Darmstadt (DE)

(21) Appl. No.: 11/912,278

(22) PCT Filed: Apr. 24, 2006
(86) PCT No.: PCT/EP06/03735

§ 371 Date: Sep. 26, 2008

(30) Foreign Application Priority Data
Apr. 22, 2005 (DE) 10 2005 018 731.5

(51) Int. Cl. B44C 1/22; A61F 2/82; A61B 17/58; A61F 2/01

(52) U.S. Cl. 216/41; 623/1.44; 606/60; 606/200

(57) ABSTRACT

A process for producing a self-supporting layer made of a titanium and nickel alloy with superelastic and/or shape memory properties has the following steps: a substrate entirely or at least mainly made of silicon is provided, a layer of said alloy is applied to a surface of the substrate, the substrate with the desired form is cut out of a wafer or formed by a wafer with the desired form; at least some zones of the lateral surfaces of the substrate adjoining the zones of the surface of the substrate which receive the layer are subjected to an etching process; a layer of said alloy is applied to the surface of the substrate; and the substrate is removed from the applied layer. Also disclosed is a substrate suitable for carrying out the process and an object, in particular an implant, comprising at least one layer produced by this process.

The present invention relates to a method for producing a self-supporting layer made of an alloy comprising titanium and nickel, the layer having superelastic behaviour and/or shape memory properties. Self-supporting layers of this type, also referred to as self-supporting films, can, in particular, be used as a biocompatible implant, for example as an embolism filter or as straps or generally as connecting members between the bones of the human skeleton. After a material-uniting connection, in particular after welding or adhering to a pipe, layers of this type can also be inserted into blood vessels as stents. The invention therefore also relates to an article, in particular an implant, comprising at least one layer produced by this method. The invention also relates to a substrate which is suitable for carrying out the method.

Materials having shape memory properties (SM materials) are distinguished, in particular, in that they can be deformed in a low-temperature phase with a martensite structure and, after subsequent heating in a high-temperature phase with an austenite structure, remember and re-assume this impressed shape. A frequently utilised property of materials of this type is their superelastic behaviour. Within a specific temperature range above characteristic pre-stress, which can be a few hundred MPa, a plateau occurs in the stress-strain curve. In this strain range, the austenite is converted into martensite. In accordance with the stress applied, the stress-induced martensite can be detwinned and thus allows within the plateau deformation of the material under a constant counterforce. In this case, strains of up to approx. 8% can be applied via the phase transformation into the stress-induced martensite without the occurrence of plastic deformation. When the load acting on the martensite is relieved, the martensite is converted back with hysteresis or the plateau stress into the starting state of the austenite.

Materials made of nickel-titanium alloys (NiTi) are often used in medical engineering on account of their good biocompatibility. The superelastic properties of the nickel-titanium alloys are advantageous in medical tools such as catheters which are used, for example, for positioning stents and are exposed to strong deformations when inserted into the body. Tissue spreaders having superelastic properties have the advantage of damaging the tissue less than spreaders made of other materials. In addition, the shape memory effect can be utilised in implants such as stents or embolism filters. In this case, the implants are deformed in the martensitic state at room temperature. Subsequently, the deformed implants are inserted into the body where the high-temperature phase of austenite is stable at body temperature. The implant is then converted and remembers its original shape. The folded-up stents and embolism filters are thus able to unfold automatically.

In principle, the nickel content of the alloy used for producing the layer can be varied, depending on the application, within broad limits of between 2 and 98 atom %. Preferably, however, it is proposed that the nickel content of the alloy be between 45 and 60 atom %.

In the past, thin shape memory layers having superelastic behaviour have conventionally been produced using physical deposition methods, preferably by cathode atomisation or sputtering. In this case, for the production of crystalline layers, either deposition has to be carried out onto a heated substrate at least 400° C. or, following the sputtering process, solution annealing has to be carried out at approx. 500-800° C. A drawback of this is that an additional sacrificial layer is required to produce self-supporting layers. In order to obtain self-supporting nickel-titanium films, there is applied before the deposition of the nickel-titanium alloy a sacrificial layer which, after the application of the nickel-titanium alloy, has to be removed using a wet-chemical method, so the nickel-titanium film does not contain any of the substrate. The two additionally required method steps of applying and removing the sacrificial layer increase the complexity of the production method and thus take up more time and increase production costs. A further drawback of using a sacrificial layer is that, if heated substrates are used, diffusion can lead to blending of the sacrificial layer with the applied nickel-titanium layer. However, the change in the composition of the nickel-titanium layer markedly influences the properties of the alloy. Thus, for example, the conversion temperature can change and adversely affect the superelasticity. Impurities caused by the sacrificial layer might also restrict the biocompatibility of the nickel-titanium layer. This can lead to the nickel-titanium films produced in this way being unusable for the intended purposes.

A further crucial criterion for the nickel-titanium layers produced in this way is their strength. Depending on the intended use, specific minimum strength values can be prescribed for nickel-titanium layers. In the past, in the case of thin layers of nickel-titanium alloys, relatively high breaking strength of 1,200 MPa was achieved only using a complex and very expensive production method known from US 2003/0059640 A1 as the “ABPS method”. This method requires a very expensive coating system which has to be specifically designed, wherein the compulsory cooling of the target material has to be deactivated during coating. As a result, the substrate and the nickel-titanium layer become very hot during the coating process, so the samples subsequently have to be quenched with a great deal of effort so as to maintain a homogeneous, supersaturated mixed state in order to be able to carry out subsequent controlled ageing. In this case, there is also required for producing self-supporting nickel-titanium layers a sacrificial layer which in a subsequent process has to be removed using a wet-chemical method, and this leads to the aforementioned drawbacks.

An especially smooth substrate surface is crucial for achieving high breaking strength. If crack nuclei in the form of notches or pores are formed during the production of the layer, the material fails in a tensile test at stresses much lower than the theoretical breaking strength. There are then achieved in the material local stress peaks which exceed the breaking strength limit. Stress peaks of this type are produced at notches, such as pores in the interior and scratches at the surface, by concentration of stress. The complex ABPS method known from US 2003/0059640 A1 therefore also seeks to achieve a substrate surface which is as smooth as possible.

The object of the present invention is to provide a method of the type mentioned at the outset that is simple to carry out and using which self-supporting nickel-titanium layers having very high breaking strength can be produced especially rapidly and cost-effectively.

According to the invention, this object is achieved by a method according to claim 1. Advantageous configurations and developments of the invention emerge from the dependent claims.

It is fundamental to the solution according to the invention that the production method includes the following method steps: A substrate, which at least predominantly contains silicon or preferably consists entirely of silicone, is prepared for the application of a layer of said alloy to one of its surfaces. In this case, the substrate is either cut out of a wafer in the desired shape or formed by a wafer which is already provided in the desired shape. At least those regions of the lateral faces of the substrate that adjoin the regions of the surface of the substrate that receive the layer to be applied are subjected to an etching process before a layer of said alloy is applied to the surface of the substrate. The substrate is subsequently removed from the layer thus applied which is then available as a self-supporting layer or as a self-supporting film.

In this way, there is provided a method which can be carried out rapidly and inexpensively and allows the production of self-supporting nickel-titanium layers having superelastic behaviour and/or having shape memory properties even without the use of a sacrificial layer. Problematic blending of the sacrificial layers, for example of gold, copper, chromium or iron-cobalt layers with the nickel-titanium layer, as a result of diffusion processes is therefore reliably ruled out even if heated substrates are used. The face of contact between the silicon substrate and the nickel-titanium layer generally does not present any problems owing to the respectively provided surfaces (TiO₂ and SiO₂). The nickel-titanium films applied in accordance with the invention can easily be mechanically detached from the substrate.

A further fundamental advantage of the method according to the invention is that there can be achieved, despite the simplicity of the method, especially high strength values of the nickel-titanium layers that in the past could be achieved only with the very high costs described. Experimental investigations using the tensile test produce, for the production method simplified in accordance with the invention, maximum stresses at break of the nickel-titanium layers of 1,200 MPa at a strain of 11.5%. These values thus correspond to the stresses at break and strains of the layers produced using the complex ABPS method described in US 2003/0059640 A1.

In order to achieve in a simplified manner especially high strength of the nickel-titanium layer, it is fundamental to the method according to the invention that not only the substrate surface onto which the layer is deposited but also the edges which delimit the surface and form the contact between the surface and the lateral faces of the substrate have an especially smooth composition. An especially high-quality substrate can be obtained in this way. The quality of the substrate, having a surface which is as smooth as possible and having edges which are as smooth as possible, and controlled coating parameters are crucial in the production of self-supporting nickel-titanium layers by sputtering. Extremely smooth edges of the substrate can be produced using the method according to the invention. Edge roughness of only approx. 100 nm or less can thus, for example, be achieved. This allows crack nuclei in the form of notches at the edges to be avoided as early as during the process for production of the layer.

The use according to the invention of such high-quality substrates can also easily replace the frequently used method of electropolishing to improve the edge roughness of tensile samples.

Preferably, at least those regions of the substrate that are subjected to an etching process are opened before the etching. In order to open the substrate regions to be etched, oxide layers (SiO₂ surfaces) are, in particular, removed, for which purpose hydrofluoric acid can advantageously be used.

It is particularly advantageous if the substrate is cut out of a wafer in the desired shape using a suitable etching mask. In this case, the etching process is at the same time also the step of cutting the substrate out of the wafer, so two partial steps of the method according to the invention can advantageously be carried out simultaneously, and this further simplifies the method and further shortens the time required to carry out the method.

According to a particularly preferred embodiment of the invention, provision is in this case made for a resist, in particular a photoresist layer, to be applied to the wafer, the resist subsequently being prestructured to form an etching mask in a lithography process using a lithography mask corresponding to the shape provided for the substrate and an exposure source, and the etching process being carried out after this prestructuring of the resist. Using this photolithographic method advantageously allows a plurality of substrates to be cut out of a wafer in a single etching process.

According to an alternative embodiment of the invention, the substrate can also be cut or sawn out of a wafer, the cut faces of the substrate that are thus produced subsequently being subjected to the etching process. The substrate can in this case be cut out of the wafer, for example, in a manner known per se by laser cutting or using a diamond-coated saw, also referred to as a wafer saw.

The etching process can be carried out particularly simply and cost-effectively by a wet etching method, a KOH solution preferably being used. However, in principle, a dry etching method can also be used.

In the medical field, in particular, a metal foil produced in accordance with the invention can be used in an especially versatile manner if the alloy layer is applied to the substrate at a thickness of between 0.5 μm and 200 μm, in particular between 2 μm and 100 μm. A particularly preferred range of the thickness of the alloy layer is between 5 μm and 50 μm.

According to a particularly preferred embodiment of the method according to the invention, the nickel-titanium layer is deposited onto the substrate by sputtering, in particular by magnetron sputtering. Sputtering is known per se for the production of thin layers using cathode atomisers. In this case, gas ions strike with high energy the sputter target which is made of the material from which the layer to be applied is to be produced. Physical pulse and energy transmission enables the gas ions to strike from the target atoms which fly toward the material to be coated, which is referred to as the sputter substrate, i.e. in the present application toward the silicon substrate, where they produce the desired coating.

The use of sputtering allows the thickness of the deposited alloy layer to be set very precisely. In the method according to the invention, sputtering also allows highly controllable and especially uniform distribution of the titanium or nickel contents, thus allowing a local increase in the nickel concentration or nickel-rich phases, which cannot be ruled out in other methods, to be avoided. There is therefore no risk of the metal foil produced in accordance with the invention not being admitted for application in the medical field owing to possible allergic reactions resulting from inadmissibly high concentrations of nickel.

A particularly dense structure of the applied nickel-titanium layer can be achieved in that the deposition temperature is at least 400° C., preferably at least 450° C. In this case, a recrystallised structure in Zone 3 of the Thornton diagram can be achieved. As suitable sputtering parameters, it is furthermore proposed to set the sputtering pressure to at least 2.3 μbar, the sputtering power preferably being at least 500 W. In the selection of suitable sputtering parameters, there is no need to use a sacrificial layer for producing self-supporting layers, even if oxidised silicon substrates are used, as the applied nickel-titanium layer can easily be detached from the substrate mechanically, in particular using pointed forceps or a scalpel, if appropriate assisted by ultrasound.

It is particularly advantageous if the etching process, which according to the invention is provided before the application of the layer, also includes at least those edges of the substrate that are located between the regions of the surface of the substrate that receive the layer and the adjoining lateral faces of the substrate. This provides an especially smooth composition of these edges and thus a particularly high-quality substrate, and this in turn leads to the advantageous particularly high strength values of the self-supporting nickel-titanium layers to be applied. Etching of the back, remote from the surface to be coated, of the substrate is in this case not required and also does not lead to the desired results and advantages without etching of the substrate regions provided in accordance with the invention.

The present invention also relates to a substrate for carrying out the above-described method, wherein the substrate at least predominantly contains silicon or consists entirely of silicon and wherein at least those regions of the lateral faces of the substrate that adjoin those regions of the surface of the substrate that receive the layer to be applied are etched. A substrate of this type can advantageously be used a plurality of times for the application of nickel-titanium layers.

In addition, the present invention relates also to articles which have superelastic behaviour and/or shape memory properties and comprise at least one layer produced by the method of the type described hereinbefore. An article of this type may preferably be an implant for the human body, in particular a stent or an embolism filter. Furthermore, articles of this type can also be used as connecting members, for example as straps between bones of the human or an animal skeleton.

Further advantages and features of the invention will emerge from the subsequent description given with reference to the figures, in which:

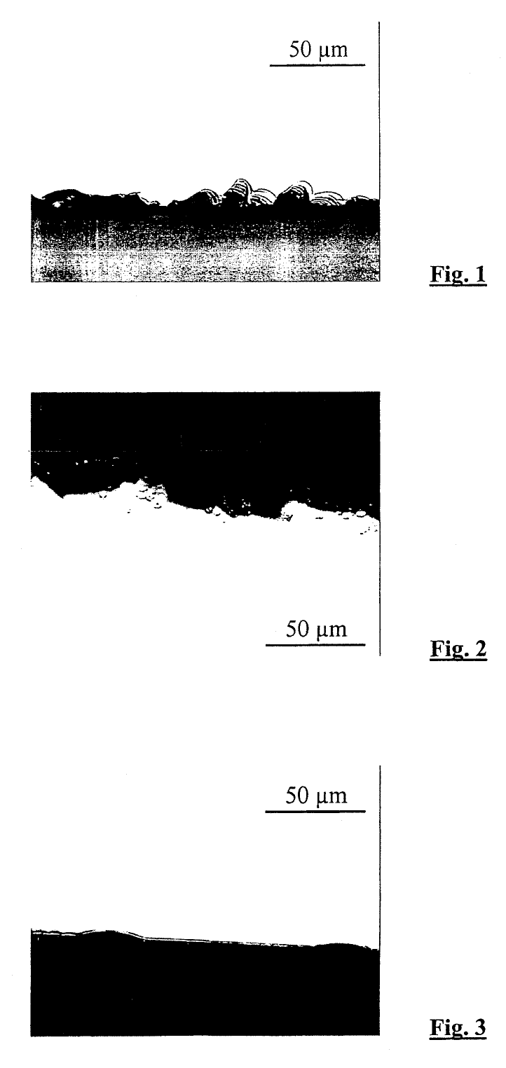

FIG. 1 is a microscope image of a nickel-titanium layer on a silicon substrate which was cut up using a wafer saw;

FIG. 2 is a microscope image of a nickel-titanium layer on a silicon substrate which was cut up by laser cutting;

FIG. 3 is a microscope image of a nickel-titanium layer on a silicon substrate which was cut up in accordance with the invention by KOH etching; and

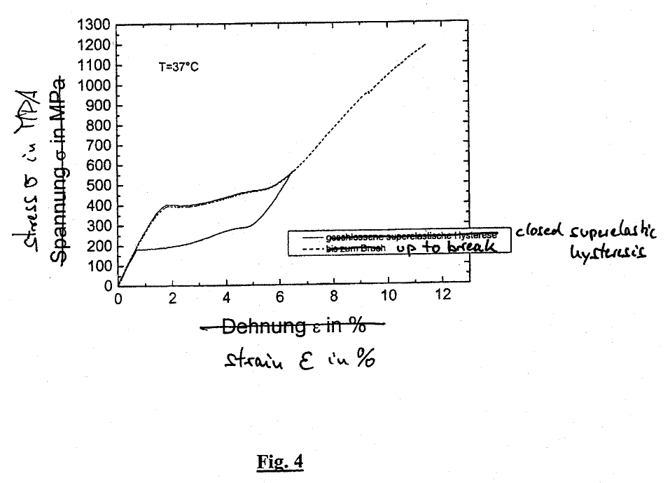

FIG. 4 shows the stress-strain curve of a nickel-titanium layer produced in accordance with the invention.

FIGS. 1 to 3 are micrographs of a substrate coated with a nickel-titanium layer, the coated surface of the substrate being parallel to the drawing plane. These micrographs clearly reveal that the method according to the invention (FIG. 3) can be used to achieve a much smoother edge or lateral face of the substrate. Whereas in FIGS. 1 and 2 the edges or lateral cut faces are shown as a contour with clearly visible waves and indentations or notches, the edge or lateral face, produced in accordance with the invention, of the substrate in FIG. 3 is formed by an almost rectilinearly extending line. In this case, the lateral face of the substrate that in FIG. 3 is located perpendicular to the drawing plane and the other lateral faces of this substrate are subjected over their entire surface area to an etching process. The use of such a high-quality substrate, which is distinguished not only by the particularly smooth surface of the silicon wafer out of which this substrate was cut but also by particularly smooth lateral faces, allows nickel-titanium layers having particularly high breaking strength to be produced in an especially simple manner.

The stress-strain diagram, shown in FIG. 4, of a nickel-titanium-layer sample produced in accordance with the invention shows with the solid line closed superelastic hysteresis. The broken line shows the further behaviour of the sample up to the break at stress of approx. 1,200 MPa.

What is claimed is:

1. Method for producing a self-supporting layer made of an alloy which comprises titanium and nickel and has at least one of superelastic behavior or shape memory properties, including the following method steps:
a substrate which at least predominantly contains silicon or consists entirely of silicon is provided for the application of a layer of said alloy to a surface of the substrate, wherein the substrate is one of cut out of a wafer in the desired shape or formed by a wafer which is provided in a desired shape;
at least those regions of the lateral faces of the substrate that adjoin the regions of the surface of the substrate that receive the layer are subjected to an etching process;
a layer of said alloy is applied to the surface of the substrate; and
the substrate is removed from the applied layer.

2. Method according to claim 1, wherein at least those regions of the substrate that are subjected to an etching method are opened beforehand.

3. Method according to claim 2, wherein, for opening those regions of the substrate that are to be etched, oxide layers are removed.

4. Method according to claim 1, wherein the substrate is etched in the desired shape out of a wafer, using an etching mask.

5. Method according to claim 4, wherein a resist is applied to the wafer, in that the resist is prestructured to form an etching mask in a lithography process using a lithography mask corresponding to the shape provided for the substrate and an exposure source, and wherein the etching process is carried out after the prestructuring of the resist.

6. Method according to claim 1, wherein the substrate is cut or sawn out of a wafer and in that the cut faces of the substrate are subsequently subjected to the etching process.

7. Method according to claim 1, wherein, in the etching process, a wet etching method is carried out.

8. Method according to claim 1, wherein the layer of said alloy is applied to the substrate at a thickness of between 0.1 μm and 500 μm.

9. Method according to claim 1, wherein the layer of said alloy is applied to the substrate by sputtering.

10. Method according to claim 9, wherein the deposition temperature is at least 400° C.

11. Method according to claim 1, wherein at least those edges of the substrate that are located between the regions of the surface of the substrate that receive the layer and the adjoining lateral faces of the substrate are subjected to an etching process before the layer is applied.

12. Substrate for carrying out the method according claim 1, wherein the substrate is made at least predominantly of silicon and wherein at least those regions of the lateral faces of the substrate that adjoin the regions of the surface of the substrate that receive the layer to be applied are etched.

13. Article having superelastic behaviour and/or having shape memory properties, comprising at least one layer produced by the method according to claim 1.

14. Article according to claim 13, wherein it is an implant for the human body.

15. Method according to claim 3, wherein in removing the oxide layers, using hydrofluoric acid.

16. Method according to claim 5, wherein the resist applied to the wafer is a photoresist layer.

17. Method according to claim 7, wherein in wet etching of at least those regions of the lateral faces of the substrate that adjoin the regions of the surface of the substrate that receive the layer, using a KOH solution.

18. Method according to claim 8, wherein the layer of said alloy is applied to the substrate at a thickness of between 1 μm and 100 μm.

19. Method according to claim 8, wherein the layer of said alloy is applied to the substrate at a thickness of between 5 μm and 50 μm.

20. Method according to claim 10, wherein the deposition temperature is at least 450° C.

21. Article according to claim 13, wherein the implant is one of a stent, an embolism filter, or a connecting member between bones.
